(19) United States
(12) Patent Application Publication
(10) Pub. No.: US 2024/0100293 A1
(43) Pub. Date: Mar. 28, 2024

(54) A MEDITATION DEVICE

(71) Applicants: Navneet Kumar RATHORE, Indore (IN); Prachi RATHORE, Indore (IN)

(72) Inventors: Navneet Kumar RATHORE, Indore (IN); Prachi RATHORE, Indore (IN)

(21) Appl. No.: 18/271,484

(22) PCT Filed: Dec. 26, 2021
(86) PCT No.: PCT/IN2021/051207

§ 371 Date: Jul. 10, 2023

(51) Int. Cl. A61M 21/02

(52) U.S. Cl. CPC A61M 21/02; A61M 2021/0022

(57) ABSTRACT

The present invention relates to a meditation device; wherein the meditation device comprising of a vibrator (E), a sound producing tool (F), and optionally a light source connected to a circuit board (D) and are enclosed in a ‘housing’ (A).

### FIELD OF THE INVENTION

The present invention relates to a device. Particularly, the invention relates to a meditation device.

### BACKGROUND OF THE INVENTION

Meditation has been a well-known art, performed since ancient time by various practitioners. Different techniques of meditations have been developed and practiced in the art to attain the state of meditation.

Some of the techniques practiced in the art are focusing on breathing, various chakras of body, technique of focusing on certain object such as tratak, noticing own thoughts, various chanting practices and the like. All these techniques have been practiced with an aim of attaining mindfulness, calmness, alertness and state of meditation.

Few devices have also been tried in the art to assist a practitioner of meditation to attain the status of meditation.

One system and method for customized meditation has been disclosed in Indian patent application No. 201741036892. The application discloses a system and method for customized meditation. The system includes a head gear component, a computing device component and an audio circuit component. The method includes receiving a plurality of brainwave signal data obtained from a plurality of electrode sensors located on a head gear component, processing the plurality of brainwave signal data to calculate a meditation level, processing the meditation level to calculate a length of a chant, and processing a real-time generation of the chant based on a calculated length of the chant.

Further, there are few prior art references which describe methods to measure the level of meditation based on brain wave signals analysis.

A research article by F. Travis et al./Consciousness and Cognition 13 (2004) 401-420 disclose a study which measure the impact of transcendental meditation on brainwave patterns and other physiological parameters. EEG and ERP patterns were compared across three groups of individuals distinguished by their self-reported experience of the integration of pure self-referral awareness with waking and sleeping.

Another research article by Braboszcet. Al. titled “Increased Gamma Brainwave Amplitude Compared to Control in Three Different Meditation Traditions”, Published in PLOS ONE|DOI:10.1371/journal.pone.0170647 on Jan. 24, 2017, discloses a study with the objective to examine first whether there is a consistent difference between the EEG activity of meditation practitioners compared to meditation-naive participants, and second whether different meditation practices can be distinguished based on either state or trait effects on the EEG signal.

A research article by Mandviwala et. al. published online on WWW.OALTSE.COM in April 2018, discloses a meditation analyzer system, by which a practitioner can see her/his meditation performance graphically and can compare it with previous sessions and with other individuals also.

In spite meditation has been practiced since very long time around the world and particularly in India, a meditation device which can assist a practitioner to attain the state of meditation has not been disclosed in the published prior art. Techniques known in the art are mainly focused on conventional meditation methods such as breathing meditation, body scanning method in which a practitioner has to focus on different parts of his body sequentially, focusing on different chakras of human body and the like. Accordingly, there remains a need to provide a meditation device which helps the practitioner to attain the state of meditation.

### SUMMARY OF THE INVENTION

The present invention provides a meditation device which has technically advanced over the device disclosed in the art.

In accordance with the present invention there is provided a meditation device comprising:
a. a vibrator (E),
b. a sound producing tool (F),
c. optionally a light source (G) And,
d. a circuit board/a power circuit (D).

Typically, the meditation device comprising vibrator (E), the sound producing tool (F), and optionally the light source (G) connected to each other through a circuit board (D).

Typically, the circuit board (D) comprises a chip/microcontroller (D′); wherein the chip/microcontroller (D′) regulates functioning of the vibrator (E), the sound producing tool (F) and the light source (G).

Typically, the vibrators (E), sound producing tool (F), light source (G) are enclosed in a ‘housing’ (A).

Typically, the circuit board/power circuit (D), is supplied with a power from a power supply tool; wherein the said power supply tool is a battery (B).

Typically, the meditation device adapted to function upon a power supply, wherein the power supply is controlled by a chip/microcontroller (D′) for functioning of the vibrator (E), the sound producing tool (F) and the light source (G).

In accordance with the present invention, the meditation device further comprises a Bluetooth (H), wherein the command to run the meditation device can optionally be given from a paired network or mobile device through Bluetooth pairing.

Typically, the vibrator (E) is a coin type vibration motor (E1-E20) having a diameter of 8.0 mm to 12 mm which produces a vibration force of 0.3 G to 0.8 G at a motor speed of about 10,000 RPM or more.

Typically, the sound producing tool (F) is a speaker which produces a sound having intensity of about 10 decibels to about 60 decibel and frequency in the range of about 20 Hz to about 20000 Hz, Typically, the light source (G) is a Light Emitting Diode (LED) which produces a light output of about 2 lumens to about 5 lumens.

Typically, the meditation device it produces vibration, sound, or light simultaneously or sequentially either in a continuous manner or in an intermittent manner.

Typically, the meditation device produces one of the following patterns of combination of vibration, sound and light:
i. continuous vibration, continuous sound, and optionally a continuous light.
ii. intermittent vibration, continuous sound, and optionally continuous light.
iii. intermittent vibration, intermittent sound, and optionally continuous light.
iv. intermittent vibration, intermittent sound, and optionally an intermittent light.
v. continuous vibration, intermittent sound, and optionally continuous light.
vi. continuous vibration, intermittent sound and optionally intermittent light.
vii. continuous vibration, Continuous sound, and optionally intermittent light.
viii. intermittent vibration, continuous sound, and optionally intermittent light.

Typically, the meditation device operates in a cycle of 32 minutes to 128 minutes; wherein each cycle comprises at least one phase.

Typically, the meditation device operates in a cycle of 32 minutes to 128 minutes, wherein each cycle comprises a single phase or two phases, wherein the two phases comprises phase 1 and phase 2, wherein the device produces one of the following patterns of vibration, sound and light:
i. Pattern 1: continuous vibration, continuous sound and optionally continuous light,
ii. Pattern 2: intermittent vibration wherein each repeating vibration is produced for the duration of 0.5 second to 1 seconds after a break of 0.5 second to 2 seconds between two vibrations and continuous sound and optionally intermittent light wherein each repeating light is produced for the duration of 1 minute, after a break of 1 minute between two lights,
iii. Pattern 3: intermittent vibration wherein each repeating vibration is produced for the duration of 0.5 second to 2 seconds after a break of 5 seconds to 10 seconds between two vibrations and continuous sound and optionally intermittent light wherein each repeating light is produced for the duration of 1 minute, after a break of 1 minute between two lights,
iv. Pattern 4:
   —Phase 1—Intermittent vibration wherein each repeating vibration is produced for the duration of 0.5 second to 1 seconds after a break of 30 seconds between two vibrations, continuous sound, and optionally intermittent light wherein each repeating light is produced for the duration of 1 second after a break 1 second between two lights, and,
   —Phase 2—Intermittent vibration wherein each repeating vibration is produced for the duration of 0.5 second to 1 second after a break of 15 seconds between two vibrations, continuous sound, and intermittent light wherein each repeating light is produced for the duration of 1 second after a break 1 second between two lights,
v. Pattern 5:
   a. Phase 1—Intermittent vibration wherein each repeating vibration is produced for the duration of 1 second after a break of 2 seconds between two vibrations and continuous sound and intermittent light wherein each repeating light is produced for the duration of 1 minute, after a break of 1 minute between two lights, and
   b. Phase 2—Intermittent vibration wherein each repeating vibration is produced for the duration of 1 second after a break of 1 second between two vibrations and continuous sound and optionally intermittent light wherein each repeating light is produced for the duration of 1 minute, after a break of 1 minute between two lights.

### BRIEF DESCRIPTION OF THE DRAWINGS

Understanding that these drawings depict only typical embodiments of the invention and are not therefore to be considered limiting of its scope, the invention will be described and explained with additional specificity and detail through the use of the accompanying drawings in which:

FIG. 1 illustrates schematic diagram of the meditation device disclosing its components;

FIG. 2 illustrates a front view of the two surfaces of the housing (A);

FIG. 3 illustrates a schematic diagram of a Li—Po battery;

FIG. 4 illustrates a battery (B) and its connections;

FIG. 5 illustrates a representative circuit board (D);

FIG. 6 illustrates components of microcontrollers (D′);

FIG. 7 illustrates Coin Vibration motor (E) and its components;

FIG. 8 illustrates Light source (G); A multicolor LED;

FIG. 9 illustrates schematic diagram representing the switch included in push button (J);

FIG. 10 illustrates Power Block Diagram; and

FIG. 11 illustrates position of electrodes on the head during recording.

### DETAILED DESCRIPTION OF THE INVENTION

The meditation device disclosed in the present invention is the first of its kind which is helpful to a practitioner to perform meditation and to reach the state of meditation. The device when used also shows several other associated benefits. The device is simple, lightweight, portable and easy to use. A practitioner can easily carry the device along and can use at any time according to convenience. The device is also comfortable and affordable. It can be used by any individual including children of more than 8 years of age.

The term “meditation device” as used herein is a device used to assist a practitioner, to attain the state of meditation.

The term “State of mediation” as used herein relates to a state of brain where brain waves of specific frequency are active.

In a living brain, millions of nerve cells communicate with each other by emitting brainwaves. In this way, the brain is electrically active night and day throughout one's life. The brainwaves can be measured by the method known as Electroencephalography (EEG) by placing electrodes on the scalp, amplifying the signals and displaying them on a computer monitor.

Various states of brain may go through in a day, from very relaxed to very alert. Broadly there are five different states of brain based on the brainwave activity. Activity of lower frequency brainwaves is related to relaxed state of brain. The EEG measurements can verify whether a person is in normal active state of brain, or in relaxed state of brain. In view of different research conducted in the art, generally Following 5 types of brainwaves have been identified based on their frequency.
(i) Beta waves have frequency from about 13 Hz to about 30 Hz. Waves known as high beta waves exist from approximately 25 Hz to 30 Hz and are triggered when a person is in a stressed or anxious state.
(ii) Alpha waves have frequency from about 8 Hz to about 12 Hz.
(iii) Theta waves have frequency from about 4 Hz to about 7 Hz.
(iv) Delta waves occur from about 0.5 Hz to about 3 Hz.
(v) Gamma waves have frequency from about 30 Hz to about 50 Hz.

On the basis of brainwave frequency, the state of meditation can be defined as a state of brain when the activity of alpha waves having frequency from about 8 Hz to about 12 Hz and theta waves having frequency from about 4 Hz to about 7 Hz is increased.

Table 1 explains different states of brain and corresponding brain waves

TABLE1
Different states of brain and corresponding brain waves

| Brain State | Unconscious | Meditation state | | Conscious | |
|---|---|---|---|---|---|
| Brain | Delta | Theta | Alpha | Beta | |
| waves | (Deep Sleep) | (Drowsy) | (Relaxed) | (Active) | Gama |
| Frequency | 0.5 4 8 13 30 50 | | | | |
| Band (Hz) | | | | | |

State of meditation can also be described based on pulse rate and breathing rate of a practitioner. In the state of meditation, the pulse rate and breathing remains less than 20% to 30% of the pulse rate and breathing rate in the normal state of a practitioner. In the state of meditation, the pulse rate remains in the range of 50-70 per minute and breathing rate remains in the range of 10-14 per minute. Actual pulse rate and breathing rate of a practitioner in the state of meditation may vary person to person.

The term ‘continuous manner’ as included herein relates to a pattern of vibration, sound, or light produced by the meditation device, wherein the vibration, sound, or light are produced without any break.

The term ‘intermittent manner’ as included herein relates to a pattern of vibration, sound, or light produced by the meditation device, wherein the vibration, sound, or light is produced for a specific time duration in a series of repetitions, wherein each repeating vibration, sound or light is produced for a particular time duration after a break of particular time duration between two vibrations, sounds and lights.

‘Intermittent vibration’ as used herein includes a pattern of vibration produced by the mediation device wherein vibration is produced in a series of repetitions and wherein each repeating vibration is produced for the duration of 0.5 second to 1 minute after a break of 0.5 second to 1 minute between two vibrations.

‘Intermittent sound’ as used herein includes a pattern of sound produced by the mediation device wherein sound is produced in a series of repetitions and wherein each repeating sound is produced for the duration of 0.5 second to 1 minute after a break of 0.5 second to 1 minute between two sounds.

‘Intermittent light’ as used herein includes a pattern of light produced by the mediation device wherein light is produced in a series of repetitions and wherein each repeating light is produced for the duration of 0.5 second to 10 minutes after a break of 0.5 second to 10 minutes between two lights.

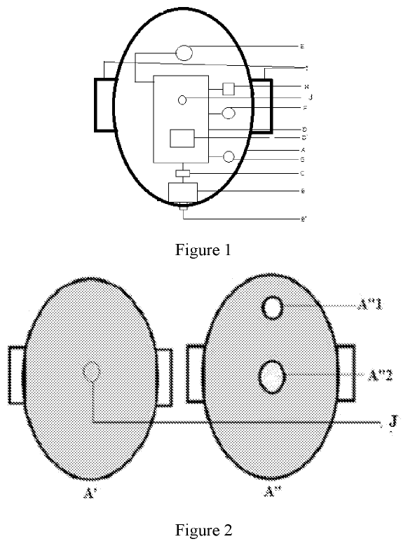

The meditation device is illustrated in accompanying FIG. 1. FIG. 1 is a schematic diagram of the device of the present invention which discloses various components of the device.

Components of device: As shown in FIG. 1 the device comprises:
A—Housing
B—Battery
B′—Battery charger point
C—Voltage regulator
D—Circuit board
D′—Microcontroller Unit/chip (MCU)
E—Vibrator
F—Sound producing tool
G—Light source
H—Bluetooth
I—Hook for belt
J—Push buttons
K—Power source

The term “chip-controlled device” as used herein relates to a meditation device wherein the functioning of different components of the device i.e. Vibrator (E), sound producing tool (F) and light source (G) in a particular pattern is regulated by a chip i.e. a Microcontroller Unit or a microprocessor (D′).

### General Layout:

The general layout of the meditation device is in a way that a single housing (A) contains all of the components of the device. The housing (A) can be made of molded plastic, or 3D printed, the size and shape of housing can vary according to the size- and shape of meditation the device. The material used for making the housing does not

harm skin and is durable, lightweight, and provide proper insulation. The housing can be of different shapes e.g. oval, circular, rectangular, square, elliptical etc. In particular, the shape of housing is oval. From its outer view, the housing has two opposite surfaces, front surface (surface A′) and rear surface (surface A″). The rear surface (surface A″) is the surface of the housing that touches skin of a practitioner when the device is in use. The front surface (surface A′) is the surface opposite to back surface (surface A″), which faces external environment when the device is in use. The two surfaces of the housing are illustrated in FIG. 2.

FIG. 2 illustrates two opposite surfaces, A′-front surface (surface A′) and A″2-rear surface (surface A″) of housing from its outer view. The front surface (surface A′) is opaque. Front surface contains a switch/push button (J) which is used to on and off the meditation device. The rear surface (surface A″) has two spaces as shown in FIG. 2 (A″). Space A″1 is a transparent part which is designed to display the light generated from a light source, when the device is functional. Space A″2 is a perforated portion in the surface A″ designed to pass the sound generated from a sound producing tool, when the device is functional. The rear surface (surface A″) either is flat or slightly convex to fit the practitioner's forehead. In one embodiment, the material of the rear surface (surface A″) is soft enough to provide extra cushioning and comfort to a practitioner.

The two opposite sides of external surface of the housing includes two hooks (I) as shown in the FIG. 1. The hooks are designed to tie two ends of a belt. The belt can be used to tie the device around head of a practitioner in such a way that the device is placed on forehead and the rear surface (surface A″) touches forehead between two eyebrows. The belt is an elastic belt that can be adjusted accordingly to comfort of a practitioner. The material of used in belt and rear surface (surface A″) is such that it does not cause any health concern e.g. skin irritation and is comfortable to practitioner.

The battery (B) as shown in FIG. 1 is included in the meditation device to supply power to all components of the device.

The major factor that could be an issue with the battery for the meditation device is how long it is able to supply power for the device before the battery (B) needs to be recharged. There are a few types of batteries that can be utilized within the design of our device. The batteries that can be used for the present device include but are not limited to Lithium Polymer (Li—Po) and Lithium-ion polymer (Li-Ion) batteries.

Power capacity of battery is defined as the amount of energy that is stored within the battery. The power capacity is normally conveyed in Watt-hours (Wh). A Watt-hour is the amount of voltage supplied by the battery, multiplied by the amount of current battery can provide per hour as shown in following formula:

Voltage (V)×Current (A)×Hours (T)=Watt-hour (Wh)

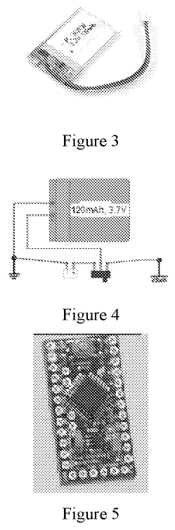

Upon detailed research, it is decided that the suitable battery for the present device would be the Lithium-Polymer (Li—Po battery). It is believed that the said battery (B) will work well within the present meditation device. There are few options of Li—Po batteries available. In choosing which Li—Po battery is most suitable for the meditation device, one of major deciding factors is the capacity of the battery. A milliampere hour (mAh) describes the capacity of energy charge that a device can operate on before having to recharge the battery. Batteries of power capacity from 90 mAh to 135 mAh are found to be suitable for the meditation device of the present invention. In particular 3.7V 120 mAh Rechargeable Lithium Polymer Battery as shown in FIG. 3 is suitable for the present meditation device.

### Specifications of the Battery

Voltage: DC 3.7V; Capacity: 120 mAh
Material: Lithium Polymer; Net Weight: 5 g
Size: 32×21×2.8 mm/1.26″×0.83″×0.11″ (L*W*T)
Connector Type: 2P PH2.0 mm Pitch; Cable Length: 5 cm/2″
Package Content: 1×Lithium Polymer Battery

FIG. 4 discloses battery and its connection en general.

Battery charger point (B′) as shown in FIG. 1 is included to charge the battery. Lithium-ion polymer (Li—Po) batteries possess certain charging requirements so that the battery can be charged efficiently. The charger we are using within our design is the Adafruit Mini Lipo with mini USB jack charger.

The battery (B) and battery charger (B′) are attached to a voltage regulator (C) as shown in FIG. 1. The system of the voltage regulator (C), battery (B) and battery charger (B′) works in unison to ensure that the components of the meditation device receive the correct amount of voltage that will remain constant for each component so that no components are damaged. Voltage regulator (C) prevents the occurrence of damaging surges and provides enough voltage that is required for a device to work. The desired function of the voltage regulator is that it works properly and maintaining an output of 3.3 Volts. The voltage regulator (C) is placed between the battery (B) and the rest of the components used in the meditation device.

The battery charger (B′) utilizes charge indicator LEDs. The indications of these LEDs are outlined in Table 2.

TABLE 2
Battery Charger Indicators

| Colour | Indication |
|---|---|
| Red | Battery is charging |
| Green | Charge cycle is complete |

Circuit Board (D) as shown in FIG. 1 is a thin sheet that has conductive etched or printed tracks on, usually made of copper, on its surface which is also known as the substrate to connect electrical components to each other, holes are placed throughout the circuit so that components that are “through hole” can be placed and soldered. All the components incorporated in the meditation device are placed on the circuit board.

A representative board is illustrated in FIG. 5:

Microcontroller Unit (D′) as shown in FIG. 1 is a mini computer that is encompassed on an integrated circuit. A basic microcontroller is comprised of the following elements:
CPU/MICROPROCESSOR (D′3)—Microcontrollers have a central processing unit which is the circuitry that is within a computer that performs instructions delegated by a computer program.
Fixed amount of memory (D′4&D′5)—Microcontrollers are built with a specified amount of ROM. RAM, or flash memory.
Inputs and output ports—These include interacting with different interfaces such as LED's LCD's or ports for USB capabilities.
Serial ports—Typical ports on microcontrollers are serial ports like universal asynchronous receiver/transmitter, also known as a UART.
Timers (D′1)—Many microcontrollers have a variety of timers used as oscillators, clock functions, or pulse generation.
ADC/DAC (D′2)—Analogue to digital converters and digital to analogue convertors are used to modify an input signal depending on a device's functionality and application.
Interpret Control—This type of controller gives delays that are necessary for a program to run optimally.

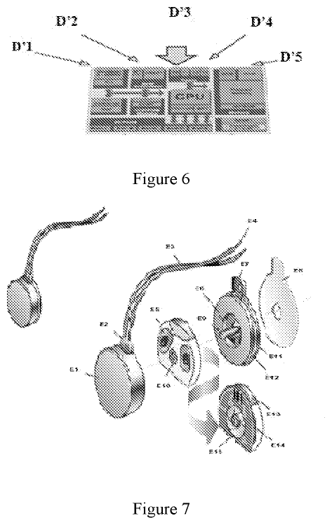

FIG. 6 shows a couple of the components (D′1-D′5) that can be found within microcontroller and shows the typical layout.

Multiple microcontrollers were researched; however, three microcontrollers were compared to determine the best fit for the meditation device. The comparisons can be seen in the Table 3.

TABLE 3
Comparison of Microcontrollers

| Name | Arduino Pro Mini | MSP430FG6626 | Raspberry Pi 3 |
|---|---|---|---|
| DC Input (V) | 3.3-12 | 3.6-5.5 V | 3.3-5 V |
| I/Os | 14 | 8 | 35 |

The Raspberry Pi 3 was ultimately chosen because of the amount of input and output ports and for easier testing purposes.

A Vibrator (E) as shown in FIG. 1 is a compact size coreless DC vibration motor used to produce vibration in a desired pattern.

The vibration motors (E) are configured in two basic varieties: coin (or flat) and cylinder (or bar). There are some components in both of their internal constructions. The vibration motor used in the meditation device of the present invention is a coin type vibration motor.

The vibration motor (E) used in the present meditation device is disclosed in FIG. 7. The coin vibration motor is comprised of MOTOR CASE (E1), UV EPDXY (E2), FLYING LEAD S (E3), BARE STRIP (E4), VOICE COIL WINDING (E5), PRECIOUS METAL BRUSHES (E6), FLEX PCB (E7), SELF ADHESIVE MOUNTING (E8), MOTOR SHAFT (E9), BEARING (E10), MOTOR CHASIS (E11), ndFeb Neodymium (E12), eccentric mass counter weight (E13), COMMUTATION CIRCUIT (E14), PRECIOUS METAL CONNECTOR (E15) wherein a weight (E13), a ring magnet, rotor with commutation points (E14) attached in the front and coils assembled on the back, and power supplied brushes (E6) attached to the ring magnet. Our coin or pancake vibration motors are all Eccentric Rotating Mass (ERM) motors.

The vibration motor is shaftless has a diameter Ø8 mm-Ø12 mm. The vibration motors are compact and convenient to use. It integrates into many designs because it does not have external moving parts, and can be affixed in place with a strong permanent self-adhesive mounting system.

When the meditation device is switched on the commutation circuit (E14) alternates the direction of the field through the coils (E5), and this interacts with the N-S pole pairs that are built into the neodymium magnet (E12). A force is generated due to the magnetic field. This force causes the weight to displace. The repeated displacement of the weight produces a varying force which is felt as vibration.

Force of Vibration (F_{vibration})=Frequency/Force

F_{vibration}=m×r×w²

m—mass of eccentric weight
r—mass' offset distance
w—speed of motor (rads⁻¹)
w—2 πf

The vibration motor (E) used in the meditation device of the present invention produces a relatively low vibrational force of 0.4 G. The motor has an operating voltage range of 2.3 to 3.3 V_{DC}. At 3 V the motors speed is 10,000 (minimum) RPM with an average current consumption of 48 mA.

It is important to note that in coin vibration motor (E) the vibration amplitude and vibration frequency are inextricably linked. The magnitude of the wave is the amplitude, but the period is the vibration frequency. When the magnitude increases, the period shortens (=increased frequency).

The full term ‘Maximum Start Voltage’ is the lowest voltage that you can apply to the motor and still be sure that it will start. Coin vibration motor used in the meditation device of the present invention has a start voltage around 2.3v.

Sound producing tool (F) as shown in FIG. 1 is a speaker generally used in earphones. The speaker used in the meditation device can produce a sound of 20 Hz to 20000 Hz.

The sound produced by the speaker used by the meditation device have intensity of 10 decibel to 60 decibel and frequency in the range of 20 hrtz to 20000 hrtz.

Intensity is defined as the power per unit area carried by a wave. Power is the rate at which energy is transferred by the wave.

In equation form, intensity I is

I=P/A,

where P is the power through an area A.

The SI unit for I is W/m².

Sound intensity levels are quoted in decibels (dB) much more often than sound intensities in watts per meter squared. Decibels are the unit of choice in the scientific literature as well as in the popular media.

If silence is 0 dB, then

A Whisper: 15-25 dB

Home/office background noise: 40-60 dB

Normal speaking voice: 65-70 dB

Lawnmower: 90 dB

Power Handling: Specified in Watts (W), the power handling specification of a speaker indicates how much power a speaker can bear without causing any damage. The speaker used in the meditation device of the present invention works on a power supply of 5 mW to 20 mW at an operating voltage of 3V-3.4V with a current flow of mA to 30 mA.

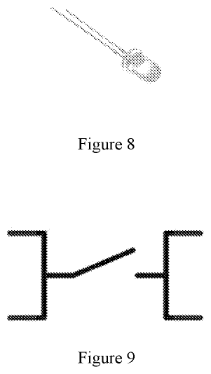

Light source (F) as shown in FIG. 1 is a common Light Emitting Diod (LED). The LED used in the present meditation device is shown in FIG. 8. The LED features 5 mm diameter head, 2 pin terminals for reliable connection. It can give out chosen color light when powered on. The color is that can be produced from the LED is selected from red, blue, green, white, and yellow. The LED requires a forward Voltage from 3.0V to 3.4V at maximum current of 20 mA. In particular 10 mA or 15 mA are suitable values for many circuits. The LED produces a light output of around two or three lumens for an input of 100 mW—equivalent to 20 or 30 lumens per watt. Alternatively, the light source can be a simple electric bulb of appropriate power capacity.

Bluetooth (H) as shown in FIG. 1 is a wireless standard for sending and receiving data over short wavelengths and distances from fixed network and mobile devices. Bluetooth operates at the frequencies between 2402 and 2480 MHz, or 2400 and 2483.5 MHZ including guard bands 2 MHz wide at the bottom end 3.5 MHz wide at the top. Bluetooth use radio technology called the frequency hopping spread spectrum, the data divides transmitted data into packets, and transmits each packet on one of 79 designated channels.

TABLE 4
Bluetooth Versioning Comparisons

| Bluetooth Version | Maximum Speed | Maximum Range |
|---|---|---|
| 3.0 | 25 Mbit/s | n/a |
| 4.0 | 25 Mbit/s | 200 feet (60 m) |
| 5 | 50 Mbit/s | 800 feet (240 m) |

Bluetooth Pairing: Implantation of Bluetooth of the pairing the two devices establish a relationship by creating a shared secret know a link key. When both devices store the same link key, they are considered bonded or paired. Then when a divide wants to communicate only with a bonded device can authenticate the identity if the other device. Link key is generated, an authenticated and Asynchronous Connection-Less between devices may be encrypted to protect changed data against eavesdropping.

System Testing: Different components of the meditation device were tested for their optimum functioning. These tests were mainly conducted to assess if the components of the meditation device are well connected and what is the optimum voltage and current requirement for their optimum functioning. Following tests were conducted:
1. Bluetooth Testing: The microcontroller unit was used to test the Bluetooth components.
2. Vibration motor testing: The design to control the vibration motor was then tested.
3. Power Testing: The power system testing includes the voltage regulator, battery, and battery charger testing. The battery and regulator were also hooked up to components to test the power circuit.
4. Voltage Regulator Testing: A simple test on the voltage regulator was done to ensure that it was working as specified in the datasheet.
5. Speaker testing
6. LED testing

Push button (J) as shown in FIG. 1 and FIG. 2A′, is provided in the meditation device. The push button (J) can be seen on surface A′ of the meditation device as shown in FIG. 2A′. The push button (J) is essentially a switching type instrument that controls switching on and off the meditation device. It includes a biased switch as shown in FIG. 9, which is normally in its set position and returns to that position once engaged. The switch includes an instrument that returns the button to its original position once the button is pushed then released by the operator. When the push button (J) is pressed, contact is made and current start flowing into the electric circuit of the meditation device. When the push button (J) is released, contact is broken and current flow stops. Push button is composed of some type of firm material such as metal or plastic. It possesses a flat shape and is suited to adapt to the human hand or finger so that the button can be easily operated.

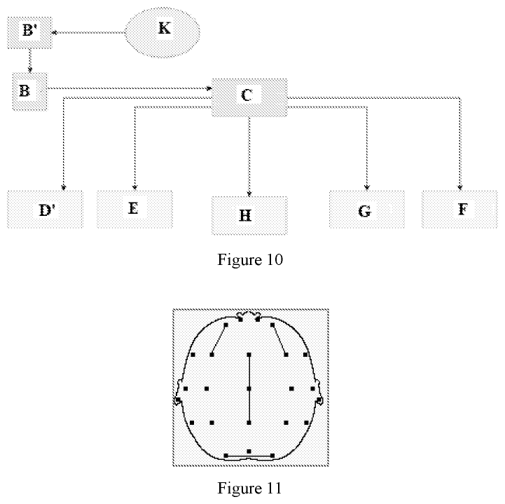

Different components of the meditation device as shown in FIG. 1 and as described in above description, are connected through a power circuit as shown in power block diagram in FIG. 10.

Power Consumption: Maximum power consumption by different components of the meditation device is shown in Table 5.

TABLE 5
Device current consumption

| Component | Max Current Draw | |
|---|---|---|
| Microcontroller (D′) | 150 | mA |
| Bluetooth (H) | 50 | mA |
| Vibration motor (E) | 50 | mA |
| Speaker(F) | 30 | mA |
| LED(G) | 20 | mA |
| Total | 300 | mA |

All the components incorporated in the meditation device are placed on the circuit board (D). The battery (B) is positioned on one side of the circuit board (D) while other components i.e. vibration motor (E), speaker (F), LED (G), and Bluetooth (H) are positioned on the other side of the circuit board (D). All components of the meditation device are kept in the single housing (A).

Smart phone application: The meditation device of the present invention is a smart phone application (smart phone App) controlled device wherein the smart phone smart phone App is specifically designed for the meditation device. In order to run the meditation device, it is first required to be paired with a smart phone through Bluetooth pairing. Once the meditation device is paired with the smart phone, command to run the meditation device on a selected mode can be given through smart phone App.

Functioning of the meditation device: Functioning of the meditation device is controlled through a smart phone application, specifically designed to run the meditation device. To run the device a practitioner first needs to install the smart phone application in his smart phone. Once the mobile application is installed, the meditation device is required to be pared with the smart phone through Bluetooth. Once the device is paired with a smart phone, command for functioning of the meditation device is given through the smartphone mobile application. The practitioner can select any one of the modes amongst the modes provided in the smartphone mobile application. Once the mode is selected and the command to run the device is given through the smartphone mobile application, the device will start functioning in a particular pattern of the vibration, sound and light set for the selected mode.

The meditation device can be switched on by pressing the push button (J). Once the push button (J) is pressed the device is start running in a default mode or previously selected mode. The device can be switched off by again pressing the push button (J).

The meditation device functions in any of the following different modes wherein each mode has specific pattern of vibration, sound and light:

Device functions in a cycle of 64 minutes, and each cycle comprises either one phase of 64 minutes or two phases of 32 minutes each and wherein the two phases are phase 1 followed by phase 2, and wherein when the device functions it produces one of the following patterns of vibration, sound and light:
i. Pattern 1: Continuous vibration, continuous sound and optionally continuous light.
ii. Pattern 2: Intermittent vibration wherein each repeating vibration is produced for the duration of 0.5 second to 1 seconds after a break of 0.5 second to 2 seconds between two vibrations and continuous sound and optionally intermittent light wherein each repeating light is produced for the duration of 1 minute, after a break of 1 minute between two lights
iii. Pattern 3: Intermittent vibration wherein each repeating vibration is produced for the duration of 0.5 second to 2 seconds after a break of 5 seconds to 10 seconds between two vibrations and continuous sound and optionally intermittent light wherein each repeating light is produced for the duration of 1 minute, after a break of 1 minute between two lights
iv. Pattern 4:
   a. Phase 1—Intermittent vibration wherein each repeating vibration is produced for the duration of 0.5 second to 5 seconds after a break of 0.5 second to 10 seconds between two vibrations, continuous sound and optionally continuous light, and
   b. Phase 2—Intermittent vibration wherein each repeating vibration is produced for the duration of 0.5 second to 5 seconds after a break of 0.5 second to 5 seconds between two vibrations and continuous sound and optionally continuous light.
v. Pattern 5:
   a. Phase 1—Intermittent vibration wherein each repeating vibration is produced for the duration of 0.5 second to 1 second after a break of 0.5 second to 2 seconds between two vibrations and continuous sound and optionally intermittent light wherein each repeating light is produced for the duration of 1 minute, after a break of 1 minute between two lights and
   b. Phase 2—Intermittent vibration wherein each repeating vibration is produced for the duration of 0.5 second to 1 second after a break of 0.5 second to 1 second between two vibrations and continuous sound and optionally intermittent light wherein each repeating light is produced for the duration of 1 minute, after a break of 1 minute between two lights.
vi. Pattern 6:
   a. Phase 1—Intermittent vibration wherein each repeating vibration is produced for the duration of 0.5 second to 2 seconds after a break of seconds between two vibrations, continuous sound and optionally intermittent light wherein each repeating light is produced for the duration of 0.5 second to 1 second after a break of 0.5 second to 1 second between two lights.
   b. Phase 2—Intermittent vibration wherein each repeating vibration is produced for the duration of 0.5 second to 2 seconds after a break of 5 seconds between two vibrations, continuous sound and optionally intermittent light wherein each repeating light is produced for the duration of 0.5 second to 1 second after a bread of 0.5 second to 1 second between two lights.
vii. Pattern 7:
   a. Phase 1—Intermittent vibration wherein each repeating vibration is produced for the duration of 0.5 second to 1 seconds after a break of 10 seconds between two vibrations, continuous sound, and optionally intermittent light wherein each repeating light is produced for the duration of 1 second after a break 1 second between two lights, and
   b. Phase 2—Intermittent vibration wherein each repeating vibration is produced for the duration of 0.5 second to 1 second after a break of 5 seconds between two vibrations, continuous sound, and optionally intermittent light wherein each repeating light is produced for the duration of 1 second after a break 1 second between two lights.
viii. Pattern 8:
   a. Phase 1—Intermittent vibration wherein each repeating vibration is produced for the duration of 0.5 second to 1 second after a break of 1 second between two vibrations, Intermittent sound wherein each repeating sound is produced for the duration of 0.5 to 5 second after a break of 0.5 second to 5 seconds between two sounds, and Intermittent light wherein each repeating is produced for the duration of 5 minutes to 10 minutes after a break 5 minutes to 10 minutes between two lights, and
   b. Phase 2—Intermittent vibration wherein each repeating vibration is produced for the duration of 0.5 second to 1 second after a break of 2 seconds between two vibrations, Intermittent sound wherein each repeating sound is produced for the duration of 0.5 second to 5 second having a break of 5 seconds to 10 seconds between two sounds, and optionally Intermittent light wherein each repeating is produced for the duration of 5 minutes to 10 minutes after a break of 5 minutes to 10 minutes between two lights
ix. Pattern 9:
   a. Phase 1—Intermittent vibration wherein each repeating vibration is produced for the duration of 1 second after a break of 1 second between two vibrations, Intermittent sound wherein each repeating sound is produced for the duration of 5 seconds after a break of 5 seconds between two sounds, and optionally Intermittent light wherein each repeating is produced for the duration of 10 minutes after a break 10 minutes between two lights, and
   b. Phase 2—Intermittent vibration wherein each repeating vibration is produced for the duration of 1 second after a break of 2 seconds between two vibrations, Intermittent sound wherein each repeating sound is produced for the duration of 5 seconds after a break of 10 seconds between two sounds, and optionally Intermittent light wherein each repeating light is produced for the duration of 5 minutes to 10 minutes after a break 5 minutes to 10 minutes between two lights
x. Pattern 10:
   a. Phase 1—Intermittent vibration wherein each repeating vibration is produced for the duration of 1 second after a break of 10 second between two vibrations, Intermittent sound wherein each repeating sound is produced for the duration of 5 seconds after a break of 10 seconds between two sounds, and optionally Intermittent light wherein each repeating light is produced for the duration of 5 minutes after a break 10 minutes between two lights, and
   b. Phase 2—Intermittent vibration wherein each repeating vibration is produced for the duration of 1 second after a break of 5 seconds between two vibrations, Intermittent sound wherein each repeating sound is produced for the duration of 5 seconds after a break of 5 seconds between two sounds, and optionally Intermittent light wherein each repeating light is produced for the duration of 5 minutes after a break 5 minutes between two lights.

How to use the meditation device: In order to use the meditation device a practitioner needs to tie the device around his head by using the belt attached to the hooks of the device in such a way that the device is placed on forehead and the back surface (surface A″) touches the forehead between two eyebrows. Once the meditation device is tied appropriately the practitioner needs to sit on a comfortable surface in a comfortable position. Once the command is given to run the meditation device in a selected mode the device will run in the selected pattern.

### EXAMPLES

Example 1: This example shows the study we conducted to determine the effects of the meditation device of the present invention on human subjects when device was run in selected modes. This is a pilot study completed in December 2020. The objective of the study was to determine the effects of using the meditation device on achieving the state of meditation in human subjects. To determine whether the subject during and after experimentation reaches the state of meditation, EEG measurements of the subjects were done during each experimentation session.

We recruited 6 healthy human subjects which include 5 test subjects and 1 control. One subject which is mentioned as control was tested for EEG without using the meditation device. Each one of the 5 human subjects went through two experimentation sessions, wherein in first session the device was run in Mode 1 and in second session the device was run in Mode 2. In both modes the meditation device run in a cycle of 64 minutes, and each cycle comprises two phases of 32 minutes each and wherein the two phases are phase 1 followed by phase 2.

In first day of study, all 5 subjects went through the first experimentation session and in second day, the same 5 subjects went through the second experimentation session. Each meditation session was for the duration of 64 minutes.

TABLE 6
Study Plan

| Timelines | Study |
|---|---|
| Day 1 | Control subject |
| | Subject 1; Experimentation Session 1 |
| | Subject 2; Experimentation Session 1 |
| | Subject 3; Experimentation Session 1 |
| | Subject 4; Experimentation Session 1 |
| | Subject 5; Experimentation Session 1 |
| Day 2 | Control subject |
| | Subject 1; Experimentation Session 2 |
| | Subject 2; Experimentation Session 2 |
| | Subject 3; Experimentation Session 2 |
| | Subject 4; Experimentation Session 2 |
| | Subject 5; Experimentation Session 2 |

Each subject was tested for their EEG measurement during the experimentation session.

Procedure: Subjects sat either on a blanket on the floor or on a chair for both experimentation sessions depending on their personal preference. The meditation device was tied around head by using the belt attached to the hooks of the device in such a way that the device is placed on forehead and the back surface (surface A″) touches the forehead between two eyebrows. Once the meditation device is tied appropriately, the command was given to run the meditation device in a selected mode. EEG electrodes were also placed on subject's scalp before starting the experimentation session so that EEG measurement can be started from the beginning of the session to see live brainwave patterns during the experimentation session.

In both modes, the vibration, sound and light produced by the meditation device had following specification:
i. Vibration: Vibrational force of 0.4 G, an operating voltage range 3 V the motors speed is 10,000 (minimum) RPM with an average current consumption of 48 mA
i. Sound: Intensity—40 db; Frequency 200 Hz to 20000 HZ; and on a power supply of 5 mW to 20 mW at an operating voltage of 3V-3.4V with a current flow of 20 mA to 30 mA.
   ii. Light: light output of around two or three lumens for an input of 100 mW—equivalent to 20 or 30 lumens per watt.

The meditation device in mode 1 was run in the following pattern:
a. Phase 1—Intermittent vibration wherein each repeating vibration is produced for the duration of 0.5 second to 1 seconds after a break of seconds between two vibrations, continuous sound, and optionally intermittent light wherein each repeating light is produced for the duration of 1 second after a break 1 second between two lights, and
b. Phase 2—Intermittent vibration wherein each repeating vibration is produced for the duration of 0.5 second to 1 second after a break of 15 seconds between two vibrations, continuous sound, and intermittent light wherein each repeating light is produced for the duration of 1 second after a break 1 second between two lights.

The meditation device in mode 2 runs in the following pattern:
a. Phase 1—Intermittent vibration wherein each repeating vibration is produced for the duration of 1 second after a break of 2 seconds between two vibrations and continuous sound and intermittent light wherein each repeating light is produced for the duration of 1 minute, after a break of 1 minute between two lights and
b. Phase 2—Intermittent vibration wherein each repeating vibration is produced for the duration of 1 second after a break of 1 second between two vibrations and continuous sound and optionally intermittent light wherein each repeating light is produced for the duration of 1 minute, after a break of 1 minute between two lights.

### Data Collection

EEG measurements were taken during the experimentation sessions of 64 minutes. The data were collected from 5 channels (2 electrodes make 1 channel) as shown in FIG. 11. As shown in FIG. 11, 2 Channels F3 and F4 were put on frontal lobe, 1 channel FZ was put on central lobe and 2 channels O2 and OZ were put on occipital lobes. The amplified EEG signals were transmitted to a computer, displayed on a screen and simultaneously stored on a hard disk. Subsequently, the EEG data was analyzed for distribution of delta, theta, alpha and beta rhythms in the EEG.

### Results:

TABLE 7
Experimentation Session 1 (Mode 1)

| | | | Brainwave Frequency (HZ) | | | |
|---|---|---|---|---|---|---|
| Timelines | Study | Channels | Before Session 1 | | During session | |
| Day 1 | Control | F3 | 13-16 | (Beta) | 10-11 | (alpha) |
| | Subject | F4 | 13-16 | (Beta) | 10-11 | (alpha) |
| | | FZ | 10-14 | (higher alpha and Beta) | 9-10 | (alpha) |
| | | O2 | 9-10 | (alpha) | 9-10 | (alpha) |
| | | OZ | 9-10 | (alpha) | 9-10 | (alpha) |
| | Subject 1 | F3 | 13-16 | (Beta) | 7-9 | (alpha) |
| | | F4 | 13-16 | (Beta) | 7-9 | (alpha) |
| | | FZ | 10-14 | (higher alpha and Beta) | 7-8 | (alpha) |
| | | O2 | 9-10 | (alpha) | 7-8 | (alpha) |
| | | OZ | 9-10 | (alpha) | 6-7 | (theta) |
| | Subject 2 | F3 | 13-16 | (Beta) | 8-9 | (alpha) |
| | | F4 | 13-16 | (Beta) | 7-8 | (theta, alpha) |
| | | FZ | 10-14 | (higher alpha and Beta) | 7-8 | (theta, alpha) |
| | | O2 | 9-10 | (alpha) | 6-7 | (theta) |
| | | OZ | 9-10 | (alpha) | 6-7 | (theta) |
| | Subject 3 | F3 | 13-16 | (Beta) | 6-7 | (theta) |
| | | F4 | 13-16 | (Beta) | 6-7 | (theta) |
| | | FZ | 10-14 | (higher alpha and Beta) | 6-7 | (theta) |
| | | O2 | 9-10 | (alpha) | 5-6 | (theta) |
| | | OZ | 9-10 | (alpha) | 5-6 | (theta) |
| | Subject 4 | F3 | 13-16 | (Beta) | 6-7 | (theta) |
| | | F4 | 13-16 | (Beta) | 6-7 | (theta) |
| | | FZ | 10-14 | (higher alpha and Beta) | 6-7 | (theta) |
| | | O2 | 9-10 | (alpha) | 5-6 | (theta) |
| | | OZ | 9-10 | (alpha) | 5-6 | (theta) |
| | Subject 5 | F3 | 13-16 | (Beta) | 6-7 | (theta) |
| | | F4 | 13-16 | (Beta) | 6-7 | (theta) |
| | | FZ | 10-14 | (higher alpha and Beta) | 6-7 | (theta) |
| | | O2 | 9-10 | (alpha) | 5-6 | (theta) |
| | | OZ | 9-10 | (alpha) | 6-7 | (theta) |

The significance of these changes will be discussed below. All the above mentioned results are statistically significant and therefore cannot be ascribed to coincidence.

TABLE 8
Experimentation Session 2 (Mode 2)

| | | | Brainwave Frequency (HZ) | | | |
|---|---|---|---|---|---|---|
| Timelines | Study | Channels | Before Session 1 | | During session | |
| Day 1 | Control | F3 | 13-16 | (Beta) | 10-11 | (alpha) |
| | Subject | F4 | 13-16 | (Beta) | 10-11 | (alpha) |
| | | FZ | 10-14 | (higher alpha and Beta) | 9-10 | (alpha) |
| | | O2 | 9-10 | (alpha) | 9-10 | (alpha) |
| | | OZ | 9-10 | (alpha) | 9-10 | (alpha) |
| | Subject 1 | F3 | 13-16 | (Beta) | 7-9 | (alpha) |
| | | F4 | 13-16 | (Beta) | 7-9 | (alpha) |
| | | FZ | 10-14 | (higher alpha and Beta) | 7-8 | (alpha) |
| | | O2 | 9-10 | (alpha) | 7-8 | (alpha) |
| | | OZ | 9-10 | (alpha) | 6-7 | (theta) |
| | Subject 2 | F3 | 13-16 | (Beta) | 8-9 | (alpha) |
| | | F4 | 13-16 | (Beta) | 7-8 | (theta, alpha) |
| | | FZ | 10-14 | (higher alpha and Beta) | 7-8 | (theta, alpha) |
| | | O2 | 9-10 | (alpha) | 6-7 | (theta) |
| | | OZ | 9-10 | (alpha) | 6-7 | (theta) |
| | Subject 3 | F3 | 13-16 | (Beta) | 6-7 | (theta) |
| | | F4 | 13-16 | (Beta) | 6-7 | (theta) |
| | | FZ | 10-14 | (higher alpha and Beta) | 6-7 | (theta) |
| | | O2 | 9-10 | (alpha) | 5-6 | (theta) |
| | | OZ | 9-10 | (alpha) | 5-6 | (theta) |
| | Subject 4 | F3 | 13-16 | (Beta) | 6-7 | (theta) |
| | | F4 | 13-16 | (Beta) | 6-7 | (theta) |
| | | FZ | 10-14 | (higher alpha and Beta) | 6-7 | (theta) |
| | | O2 | 9-10 | (alpha) | 5-6 | (theta) |
| | | OZ | 9-10 | (alpha) | 5-6 | (theta) |
| | Subject 5 | F3 | 13-16 | (Beta) | 6-7 | (theta) |
| | | F4 | 13-16 | (Beta) | 6-7 | (theta) |
| | | FZ | 10-14 | (higher alpha and Beta) | 6-7 | (theta) |
| | | O2 | 9-10 | (alpha) | 5-6 | (theta) |
| | | OZ | 9-10 | (alpha) | 6-7 | (theta) |

During the experimentation sessions, a significant rise of alpha and theta rhythms in the brain was observed in all five subjects. Alpha and theta activity were observed from all the channels during the experimentation sessions 1 and 2. The increase of these rhythms was greatest in the rear part of the brain (parietal regions).

All subjects were also tested for their pulse rate and breathing rate during the meditation sessions. The results are shown table 9.

TABLE 9
Pulse rate and Breathing rate during meditation sessions

| | Experimentation session 1 | | Experimentation session 1 | |
|---|---|---|---|---|
| | Minimum | Minimum | Minimum | Minimum |
| Subject | Pulse rate | breathing rate | Pulse rate | breathing rate |
| Control | 80 | 14 | 80 | 12 |
| Subject | | | | |
| Subject 1 | 60 | 14 | 60 | 14 |
| Subject 2 | 60 | 13 | 55 | 13 |
| Subject 3 | 70 | 10 | 50 | 10 |
| Subject 4 | 50 | 10 | 50 | 10 |
| Subject 5 | 55 | 12 | 55 | 12 |

As shown in table 9, the pulse rate and breathing rate were also found to be reduced in all subjects following two experimentation sessions.

The meditation device described in the present specification is simple, easy to perform, and effective in attaining the state of meditation. While using the meditation device other associated benefits such as alertness, mindfulness was also observed by the tested subjects. Other similar associated benefits of the meditation device are a relaxed mental state, better sleep, reduced pulse rate and breathing rate were also observed.

While specific language has been used to describe the invention, any limitations arising on account of the same are not intended. As would be apparent to a person skilled in the art, various working modifications may be made to the method in order to implement the inventive concept as taught herein. The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

We claim:

1. A meditation device comprising
a. vibrator (E);
b. a sound producing tool (F);
c. optionally a light 5 source (G); and
d. a circuit board/a power circuit (D)
characterized in that the vibrator (E), the sound producing tool (F), and optionally the light source (G) connected to each other through a circuit board (D), characterized in that the circuit board (D) comprises a chip/microcontroller (D′); wherein the chip/microcontroller (D′) regulates functioning of the vibrator (E), the sound producing tool (F) and the light source (G),
Wherein; the vibrator (E), the sound producing tool (F), and optionally the light source (G) are enclosed in a ‘housing’ (A) wherein the meditation device characterized in that the device produces vibration, sound, or light simultaneously or sequentially either in a continuous manner or in an intermittent manner.

2. The meditation device as claimed in claim 1, wherein the power circuit (D), is supplied with a power from a power supply tool, wherein the said power supply tool is a battery (B).

3. The meditation device as claimed in claim 1, wherein the meditation device adapted to function upon a power supply, wherein the power supply is controlled by a chip/microcontroller (D′) for functioning of the vibrator (E), the sound producing tool (F) and the light source (G).

4. The meditation device as claimed in claim 1, wherein the meditation device further comprises a bluetooth (H), wherein the command to run the meditation device can optionally be given from a paired network or mobile device through Bluetooth pairing.

5. The meditation device as claimed in claim 1, wherein;
a. the vibrator (E) is a coin type vibration motor (E1-E20) having a diameter of 8.0 mm to 12 mm which produces a vibration force of 0.3 G to 0.8 G at a motor speed of 10,000 RPM or more;
b. the sound producing tool (F) is a speaker which produces a sound having intensity of 10 decibels to 60 decibel and frequency in the range of 20 Hz to 20000 Hz, c. the light 5 source (G) is a Light Emitting Diode (LED) which produces a light output of 2 lumens to 5 lumens.

6. The meditation device according to claim 1, wherein the meditation device comprises a Bluetooth, and wherein the command to run the meditation device can optionally be given from the paired network or mobile device through the Bluetooth pairing.

7. The meditation device as claimed in claim 1, wherein when the meditation device produces one of the following patterns of combination of vibration, sound and light:
i. continuous vibration, continuous sound, and optionally a continuous light,
ii. intermittent vibration, continuous sound, and optionally continuous light,
iii. intermittent vibration, intermittent sound, and optionally continuous light,
iv. intermittent vibration, intermittent sound, and optionally an intermittent light,
v. continuous vibration, intermittent sound, and optionally continuous light,
vi. continuous vibration, intermittent sound, and optionally intermittent light,
vii. continuous vibration, Continuous sound, and optionally intermittent light,
viii. Intermittent vibration, continuous sound, and optionally intermittent light.

8. The meditation device as claimed in claim 1, wherein, the meditation device operates in a cycle of 32 minutes to 128 minutes; wherein each cycle comprises one phase or more than one phase.

9. The meditation device as claimed in claim 1, wherein, the meditation device operates in a cycle of 32 minutes to 128 minutes, wherein each cycle comprises a single phase or two phases; wherein the two phases comprising phase 1 and phase 2, wherein the device produces one of the following patterns of vibration, sound and light:
i. Pattern 1: continuous vibration, continuous sound and optionally continuous light,
ii. Pattern 2: intermittent vibration wherein each repeating vibration is produced for the duration of 0.5 second to 1 seconds after a break of 0.5 second to 2 seconds between two vibrations and continuous sound 10 and optionally intermittent light wherein each repeating light is produced for the duration of 1 minute, after a break of 1 minute between two lights,
iii. Pattern 3: intermittent vibration wherein each repeating vibration is produced for the duration of 0.5 second to 2 seconds after a break of 5 seconds to 10 seconds between two vibrations and continuous sound and optionally intermittent light wherein each repeating light is produced for the duration of 1 minute, after a break of 1 minute between two lights,
iv. Pattern 4:
a. —Phase 1—Intermittent vibration wherein each repeating vibration is produced for the duration of 0.5 second to 1 seconds after a break of 30 seconds between two vibrations, continuous sound, and optionally intermittent light wherein each repeating light is produced for the duration of 1 second after a break 1 second between two lights, and,
b. —Phase 2—Intermittent vibration wherein each repeating vibration is produced for the duration of 0.5 second to 1 second after a break of 15 seconds between two vibrations, continuous sound, and intermittent light wherein each repeating light is produced for the duration of 1 second after a break 1 second between two lights,
v. Pattern 5:
c. Phase 1—Intermittent vibration wherein each repeating vibration is produced for the duration of 1 second after a break of 2 seconds between two vibrations and continuous sound and intermittent light wherein each repeating light is produced for the duration of 1 minute, 5 after a break of 1 minute between two lights, and
d. Phase 2—Intermittent vibration wherein each repeating vibration is produced for the duration of 1 second after a break of 1 second between two vibrations and continuous sound and optionally intermittent light wherein each repeating light is produced for the duration of 1 minute, after a break of 1 minute between two lights.
